(19) United States
(12) Patent Application Publication
(10) Pub. No.: US 2005/0234517 A1
(43) Pub. Date: Oct. 20, 2005

(54) Apparatus and method for hemodynamic-based optimization of cardiac pacing

(76) Inventors: Frieder Braunschweig, Taby (SE); Barbro Kjellstrom, Minneapolis, MN (US)

Correspondence Address: MEDTRONIC, INC., 710 MEDTRONIC PARKWAY NE, MS-LC340, MINNEAPOLIS, MN, 55432-5604, US

(21) Appl. No.: 10/629,424

(22) Filed: Jul. 29, 2003

Related U.S. Application Data
(60) Provisional application No. 60/400,796, filed on Aug. 2, 2002.

(51) Int. Cl. A61N 1/18; A61N 1/20; A61N 1/22; A61B 5/02; A61N 1/378

(52) U.S. Cl. 607/6; 600/485

(57) ABSTRACT

The present invention demonstrates that continuous hemodynamic monitoring can be used to identify the optimal AV-delay in a pacemaker-treated patient with end stage heart failure. The AV-delay determines the timing of late diastolic filling in relation to the onset of ventricular contraction and the duration of diastolic filling. An optimal tuning of the AV-delay improves left ventricular filling pressures in patients with a DDD-programmed pacemaker and is particularly important in the presence of a compromised left ventricular function. It has been discovered that using the lowest ePAD pressure, an indirect parameter of the left ventricular end-diastolic pressure, as an indicator for the optimal AV interval. Importantly, measurements of the ePAD revealed the same optimal AV-delay as echocardiographic assessment of left ventricular diastolic filling by standard echocardiographic methods. Importantly, the HR determined as optimal during the acute hemodynamic test did not turn out to be optimal during daily living activities.

### CROSS REFERENCE TO RELATED APPLICATIONS

The present invention claims the benefit of provisional U.S. patent application Ser. No. 60/400,796 filed 2 Aug. 2002 having common title hereof and the contents of which are hereby incorporated by reference herein.

The present invention relates to a non-provisional U.S. application Ser. No. ______ (Atty Dkt P-9003.00) entitled, “Mechanically-based Interval Optimization for a Biventricular Pacing Engine,” invented by D. Warkentin and filed on common date herewith, the contents of which are hereby incorporated by reference herein.

### FIELD OF THE INVENTION

The present invention relates to the field of implantable medical devices. In particular, the present invention discloses apparatus and method for optimizing cardiac pacing algorithms based on hemodynamic physiologic data collected using a hemodynamic transducer implanted in a pacemaker patient. The present invention has specific utility with respect to heart failure patients suffering from related chronic symptoms.

### BACKGROUND OF THE INVENTION

Cardiac resynchronization therapy (CRT) has gained increased use as an alternative treatment in patients with drug refractory heart failure and an intraventricular conduction delay. Current biventricular pacemakers offer a number of programmable parameters that have potential impact on the hemodynamic status, such as heart rate, AV- and VV-interval and pacing mode.

In patients with compromised central hemodynamics, optimization of pacemaker algorithms may be crucial for the treatment success of resynchronization. Echocardiography and Doppler techniques are commonly used to optimize the AV-delay based on measurements of the diastolic mitral inflow pattern or the aortic time velocity integral. However, echocardiography equipment is not always easily accessible for the physician and the usefulness of a short-term evaluation of hemodynamic parameters at rest for long-term pacemaker programming in ambulatory patients is disputed. Therefore, an integrated hemodynamic sensor function may be helpful for the individual optimization of pacemaker devices used in patients with heart failure.

Continuous hemodynamic monitoring with an implanted device is technically feasible, safe and delivers accurate measurements over time. Initial reports suggest that the monitor may help to tailor diuretic and other drug treatments in patients with chronic severe heart failure.

The present invention is described with respect to a patient with end stage heart failure, implanted with both a biventricular pacemaker and a hemodynamic monitor. A prospective study was performed to evaluate if the hemodynamic monitor could be used for optimization of the AV-delay and heart rate.

With respect to pressure sensing apparatus capable of chronic in vivo operation, many devices and methodologies have been proposed and/or implemented in the prior art. In this regard, the following issued U.S. patents provide added details for several representative pressure monitoring techniques; namely: U.S. Pat. Nos. 5,368,040; 5,564,434; 6,171,252; and 6,221,024 the contents of each are hereby incorporated herein as if fully set forth herein.

### SUMMARY OF THE INVENTION

The present invention demonstrates that continuous hemodynamic monitoring can be used to identify the optimal AV-delay in a pacemaker-treated patient with end stage heart failure (HF). The AV-delay determines the timing of late diastolic filling in relation to the onset of ventricular contraction and the duration of diastolic filling. An optimal tuning of the AV-delay improves left ventricular filling pressures in patients with a DDD-programmed pacemaker and is particularly important in the presence of a compromised left ventricular function. It has been discovered that using the lowest estimated pulmonary artery diastolic pressure (ePAD), an indirect parameter of the left ventricular end-diastolic pressure, as an indicator for the optimal AV interval. Importantly, measurements of the ePAD revealed the same optimal AV-delay as echocardiographic assessment of left ventricular diastolic filling by standard echocardiographic methods (Ritter).

Importantly, the HR determined as optimal during the acute hemodynamic test did not turn out to be optimal during daily living in this patient. In the acute test a decrease of ePAD and RVDP was seen simultaneously with an increase of RVPP and maximal dP/dt at a heart rate of 90 bpm. The present invention demonstrates that continuous hemodynamic monitoring provides useful information for the optimization of hemodynamically important pacemaker algorithms such as the AV-delay, heart rate and pacing mode. In contrast to echocardiography, hemodynamic monitoring offers the potential to adjust pacemaker parameters even under the condition of exercise or during daily living. In patients with heart failure, the hemodynamic information may also be used to guide drug treatment and volume management.

Therefore, future devices designed for the use in patients with heart failure, such as traditional dual chamber pacemakers, bi-ventricular resynchronization devices, ICDs, and the like may contain a hemodynamic monitoring sensor, constituting an integrated heart failure management device.

### BRIEF DESCRIPTION OF THE DRAWINGS

FIG. 1 depicts measured hemodynamic impact of different AV-delays during biventricular pacing at a heart rate of 70 bpm (mean and SD of 5 consecutive tests).

FIG. 2 is a table depicting various hemodynamic metrics for certain pacing intervals and heart rates.

FIG. 3 depicts continuous hemodynamic monitoring during 7 weeks at different back-up heart rates in a patient with a biventricular pacemaker. Median (dark line) 6^{th} and 94^{th} percentile (light line) of Right ventricular (RV) systolic pressure (RVSP) RV diastolic pressure (RVDP), RV pulse pressure (RVPP) and RV contraction velocity (RV dP/dt). The heart rate (HR) is represented by the dotted line.

### DETAILED DESCRIPTION OF THE PRESENT INVENTION

The present invention was tested in the therapy for a 58 year-old male patient having cardiovascular risk factors that included cigarette smoking, hypertension and a family history of coronary artery disease and heart failure. In 1993 the patient suffered from an infero-lateral myocardial infarction (MI) that was treated by thrombolysis. Post infarction echocardiography revealed a moderately enlarged left ventricle (LV) with a left ventricular ejection fraction (LVEF) of 45%. The patient underwent complete revascularization by coronary artery by-pass grafting (CABG) in August 1994. In the post surgery period the patient developed symptoms of severe heart failure and the LVEF decreased to 15-20%. Medical therapy with diuretics, enalapril, carvedilol, ASA, pravastatin and digoxin led to significant clinical improvement. In May 1999 the patient was included in a clinical trial conducted on behalf of Medtronic, Inc. of Minneapolis, Minn., U.S.A. (for the Chronicle® implantable hemodynamic monitor). This trial was a study to evaluate the technical accuracy and reliability of an implantable hemodynamic monitor (IHM) over time. Three months later, in August 1999, the patient had a minor stroke. The corresponding IHM information revealed an episode of paroxysmal atrial fibrillation (AF) and anticoagulant treatment with warfarin was started. During the following eight months the patient was hospitalized three times for a troponin positive acute coronary syndrome caused by paroxysmal AF. Each time the patient could be successfully cardioverted, but the patient's clinical status deteriorated steadily. In May, 2000 echocardiography measurements showed significantly enlarged ventricles with a left ventricular end-diastolic diameter (LVEDD) of 81 mm, LVEF of about 10%, mitral insufficiency (grade 2/4) and tricuspid regurgitation (grade %). The patient was listed for heart transplantation.

Due to symptomatic bradycardia, first-degree heart block (P-Q interval of 260 ms) and a left anterior hemi-block with a QRS duration of 120 ms, a bi-ventricular pacemaker was implanted (the InSync® brand pacemaker manufactured by Medtronic, Inc.). The Chronicle® brand IHM (manufactuered by Medtronic, Inc. Model 9520) allows continuous, ambulatory hemodynamic recording using a pressure sensor placed in the right ventricular (RV) outflow tract. Heart rate (HR), activity and several pressure or pressure related parameters are measured and stored in the memory of the subcutanously implanted device. The data collection can be programmed to various follow-up periods that regulate the storage interval. In this disclosure RV systolic pressure (RVSP), RV diastolic pressure (RVDP), estimated pulmonary artery diastolic (ePAD) pressure (10,11), rate-of-change pressure (dP/dt) and HR measured both acutely (storage interval of two seconds) and ambulatory (storage interval of six minutes) are described.

Hemodynamic information from the IHM was collected at rest during test protocols including eight paced AV (PAV) intervals (110-250 ms) and seven different HR (50-110 bpm). AV-intervals were always tested at a HR of 70 bpm and HR at an AV-delay of 180 ms (paced) and 130 ms (sensed). The different AV-intervals and HR were programmed in a randomized order for 1-2 minutes each and a median of the last 30 seconds was used for the data analysis. The protocol was repeated each week over five consecutive weeks. Echo-Doppler measurements were used to assess the diastolic mitral inflow pattern. According to the Ritter method of echocardiography, the AV-delay providing complete end-diastolic filling without shortening of the diastolic filling time was considered optimal. In addition the hemodynamic impact of four different heart rates (60-90 bpm) were tested during periods of 5-14 days each while the patient was at home performing activities of daily living (ADL).

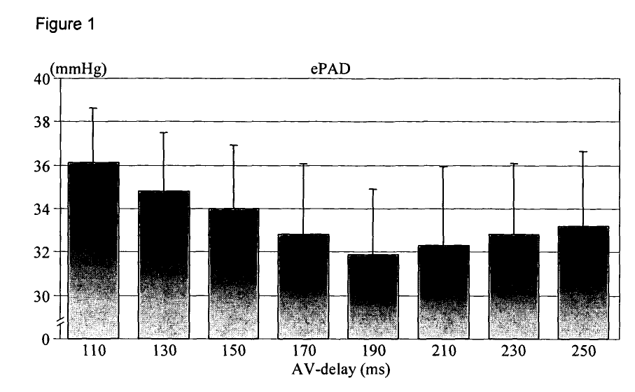

Referring now to FIG. 1, the reader can appreciate that an optimal AV delay was determined as 190 ms (PAV interval) and 140 ms (SAV interval) using the lowest obtained ePAD from the IHM as the criterion. At the optimal AV-interval the mean ePAD was 4.2 mmHg lower (31.9 mmHg) compared to the poorest setting (36.1 mmHg at 110 ms). The same PAV interval of 190 ms was determined optimal by the Echo-Doppler measurements.

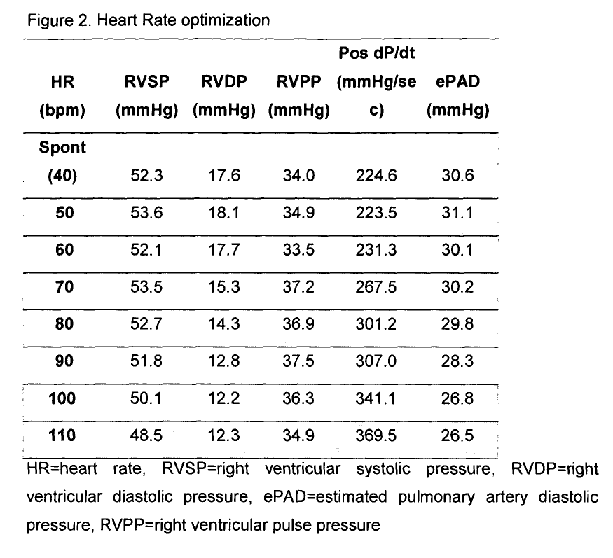

Referring now to the table set forth as FIG. 2, during the acute test of various HR an increase in right ventricular pulse pressure (RVPP) and a decrease in right ventricular diastolic pressure (RVDP) and ePAD was seen when heart rate was programmed to 90 bpm.

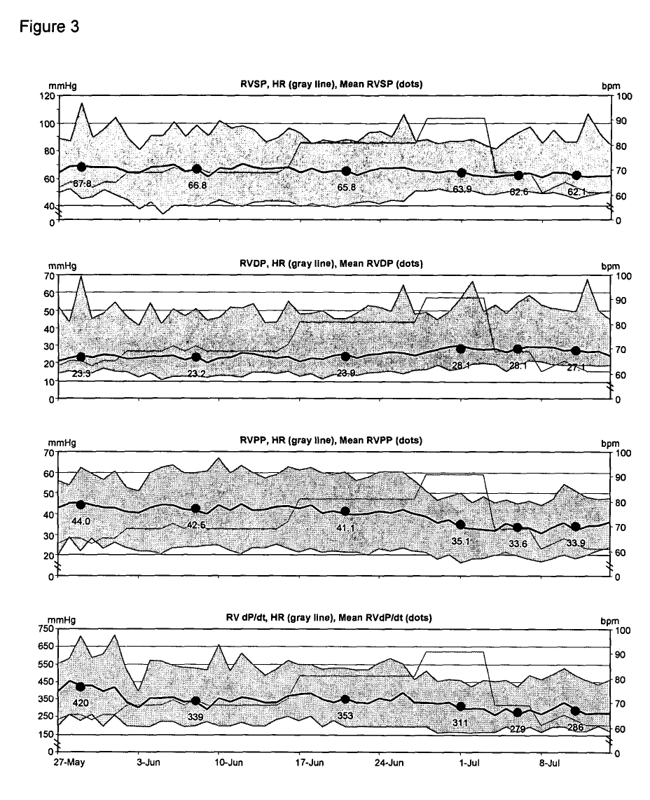

However, as depicted in FIG. 3, in an ambulatory setting, a hemodynamic deterioration was indicated by increased ePAD and RVDP and decreased pulse pressure (PP) and dP/dt when HR was programmed above 70 bpm.

The present patent disclosure demonstrates that continuous hemodynamic monitoring can be used to identify the optimal AV-delay in a pacemaker patient suffering from end-stage heart failure. The AV-delay determines the timing of late diastolic filling in relation to the onset of ventricular contraction and the duration of diastolic filling. An optimal tuning of the AV-delay improves left ventricular filling pressures in patients with a DDD-programmed pacemaker and is particularly important in the presence of a compromised left ventricular function. Therefore, we used the lowest ePAD pressure, an indirect parameter of the left ventricular end-diastolic pressure, as an indicator for the optimal AV interval.

Importantly, measurements of the ePAD revealed the same optimal AV-delay as echocardiographic assessment of left ventricular diastolic filling by the standard Ritter method. The HR determined as optimal during the acute hemodynamic test did not turn out to be optimal during performance of ADL in this patient. In the acute test a decrease of ePAD and RVDP was seen simultaneously with an increase of RVPP and maximal dP/dt at a heart rate of 90 bpm.

While ambulatory (i.e., performing ADL), a HR programmed above 70 bpm had the opposite effect. That is, increases in ePAD and RVDP and lowering of RVPP and lowering of maximal dP/dt. This indicates that an “optimal” HR as determined by a test protocol in a resting patient may help to acutely improve hemodynamics, for example in the situation of acutely de-compensated heart failure patient where an increase in HR is required to improve cardiac output (CO).

In stark contrast, however, the inventors have discovered that optimal ambulatory heart rates may only be determined during exercise or even better, while the patient performs ordinary, daily activities (i.e., ADL).

In addition, the hemodynamic impact of different VV-delays (i.e., the interval between stimulation of the right and left ventricle), and the importance of different pacing modes (for example biventricular vs. left ventricular pacing) warrants further investigation.

In this patient two devices were used. The IHM was implanted first as a part of a clinical multicenter study and the biventricular pacemaker was implanted second per clinical indications.

Accordingly, a hemodynamic sensor integrated with (or within) a biventricular pacemaker or an ICD may be implemented according to the present invention as an integrated heart failure management device. Such a device allows for recording both long-term hemodynamic trends of a patient that will help improve overall treatment of the patient, as well as for the customized hemodynamic tuning of the pacemaker/ICD operational parameters on a patient-by-patient basis.

In contrast to echocardiography, such a sensor-based optimization procedure may be performed by the pacemaker physician at any time and in the presence of a remote monitoring system even with the patient at home. Toward that end, the various data telemetry and remote patient management technologies of Medtronic, Inc. may be readily applied so that such optimization can be simply, efficiently and quickly administered irrespective of patient location.

In addition, an integrated hemodynamic sensor enables optimization of pacemaker algorithms according to the hemodynamic response during stress and during performance of the ADL. In addition, such an integrated hemodynamic sensor provides for short-term adjustments after changes of the patient's clinical condition.

The present invention demonstrates that continuous hemodynamic monitoring provides useful information for the optimization of hemodynamicly important pacemaker algorithms such as the AV-delay, heart rate and pacing mode. In contrast to echocardiography, hemodynamic monitoring offers the potential to adjust pacemaker parameters even under the condition of exercise or during daily living. In patients with heart failure, the hemodynamic information may also be used to guide drug treatment and volume management. Therefore, future devices designed for the use in patients with heart failure, such as traditional dual chamber pacemakers, bi-ventricular resynchronization devices, ICDs, and the like may contain a hemodynamic monitoring sensor, constituting an integrated heart failure management device.

While the present invention has been described with respect to a single patient and using only one illustrative embodiment, those of skill in the art to which the invention is directed will readily recognize that other related embodiments are taught hereby. The present invention is intended to cover all such embodiments as further set forth in the appended claims.

1. A system for collecting hemodynamic data from a patient and utilizing said data to optimize a cardiac pacing regimen for said patient, comprising:
a hemodynamic monitor means for continuously collecting hemodynamic data of a patient during periods of rest and periods wherein said patient is performing the activities of daily living and for storing said collected hemodynamic data;
a means for monitoring and/or stimulating cardiac tissue of a patient to provide or restore a desired cardiac rhythm; and
a means for integrating at least a portion of the collected hemodynamic data with the means for monitoring and/or stimulating cardiac tissue to optimize one or more hemodynamic characteristics of said patient.

2. A system according to claim 1, wherein the hemodynamic monitor means is a one of the following transducers, each of which provides an output signal directly or indirectly indicative of at least one hemodynamic metric of the patient:
an absolute pressure sensor adapted to be fluidly coupled to a cardiac chamber of the patient, an absolute pressure sensor adapted to be fluidly coupled to a pulmonary artery of a patient, an absolute or a differential pressure sensor adapted to be fluidly coupled to a portion of the vacsulature of a patient.

3. A system according to claims 1, wherein the means for monitoring and/or stimulating comprises a one of the following:
a pulse generator, a implantable pacemaker, an implantable cardioverter defibrillator, a muscle stimulation apparatus, an external pacemaker.

4. A system acording to claim 3, wherein the hemodynamic monitor means comprises a one of the following:
an absolute pressure sensor acapted to be fluidly coupled to a cardiac chamber of the patient, an absolute pressure sensor adapted to be fluidly coupled to a pulmonary artery of a patient, a differential pressure sensor adapted to be fluidly coupled to a portion of the vacsulature of a patient, an implantable absolute pressure sensor coupled to an external reference pressure signal; and
wherein an activity-level measurement means is optionally coupled to said patient and an output signal of said activity-level measurement means is time-synchronized to the hemodynamic monitor means and said activity-level measurement means is derived from an accelerometer transducer or a piezoelectric crystal tranducer.

5. A method of optimizing the hemodynamics of a patient having an implantable cardiac rhythm stimulation and monitoring device, comprising the steps of:
collecting hemodynamic data from said patient, during a period of time when a heart rate of the patient is elevated above a resting rate due to activity by said patient, with a hemodynamic monitor adapted to be disposed in fluid contact with a volume of venous blood of said patient;
storing said hemodynamic data in a computer readable memory medium;
collecting cardiac event data from the patient;
storing the cardiac event data in a computer readable memory medium;
analyzing said hemodynamic data in conjunction with said cardiac event data to determine a cardiac stimulation sequence intended to optimize the hemodynamics of said patient; and
providing said cardiac stimulation sequence to an implantable cardiac rhythm stimulation and/or monitoring device.

6. A method according to claim 5, wherein said hemodynamic data is at least a one of the following:
a right ventricular systolic pressure, a right venticular diastolic pressure, a pressure signal sensed in the right ventricle, an estimated pulmonary artery diastolic pressure, a pulmonary artery diastolic pressure, an estimated pulmonary artery systolic pressure, a pulmonary artery systolic pressure, a heart rate, a dP/dt value (i.e., a first derivative) of one of the foregoing pressure(s), a d²P/dt² value (i.e., a second derivative) of one of the foregoing pressure(s).

7. A method according to claim 5 or claim 6, wherein the hemodynamic data is collected substantially continuously, periodically, at a pre-determined time of day, at a pre-determined interval, while the patient is at rest, while the patient is performing typical daily activities for said patient, while the patient is strenuously exercising, and/or while the patient is exercising mildly.

8. A method according to claim 5 or claim 7, wherein during the providing step at least one of the following parameters comprises a part of the cardiac stimulation sequence: an A-V interval, a sensed-AV internval, a paced-AV interval, a V-V interval, a V-A interval, a heart rate.

9. A method according to claim 5 or claim 8, wherein the implantable cardiac rhythm stimulation and/or monitoring device comprises a bi-ventricular device.

10. A method according to claim 9, wherein said implantable cardiac rhythm stimulation and/or monitoring device is programmed to at least one of the following pacing mode(s): a dual chamber pacing mode, a ventricular pacing regime; a dual chamber sensing regime; a trigger, null and/or inhibit delay response regime (in response to a sensed cardiac event); or a rate-responsive variant thereof.

11. A method according to claim 5, claim 7 or claim 9, wherein the hemodynamic data is collected using at least one of the following data collection models:
for a set of different A-V intervals during pacing at a common heart rate,
for a first set of different heart rates using a common A-V interval, or
for a second set of different heart rates constrained in a predetermined range
for a preselected period of time.

12. A method according to claim 11, wherein, as applicable:
the set of different A-V intervals comprises a range of between of about 80 ms and about 350 ms;
the first set of different heart rates is between about 40 bpm and about 180 bpm;
the second set of different heart rates is between about 40 bpm and 180 bpm; and
the preselected period of time is between a few minutes and several days.

13. A method according to claim 12, wherein the cardiac stimulation sequence comprises data based at least in part on the lowest estimated pulmonary artery diastolic pressure measured during collection of the hemodynamic data.

14. A computer readable medium for performing a method for optimizing hemodynamics of a patient using hemodynamic data collected from said patient, comprising:
instructions for collecting hemodynamic data from said patient, during a period of time when a heart rate of the patient is elevated above a resting rate due to activity by said patient, with a hemodynamic monitor adapted to be disposed in fluid contact with a volume of venous blood of said patient;
instructions for storing said hemodynamic data in a computer readable memory medium;
instructions for collecting cardiac event data from the patient;
instructions for storing the cardiac event data in a computer readable memory medium;
instructions for analyzing said hemodynamic data in conjunction with said cardiac event data to determine a cardiac stimulation sequence intended to optimize the hemodynamics of said patient; and
instructions for providing said cardiac stimulation sequence to an implantable cardiac rhythm stimulation and/or monitoring device.
